(19) United States
(12) Patent Application Publication
(10) Pub. No.: US 2009/0181409 A1
(43) Pub. Date: Jul. 16, 2009

(54) Optical biosensor method for cell-cell interaction

(76) Inventors: Ye Fang, Painted Post, NY (US); Guangshan Li, Corning, NY (US)

Correspondence Address: CORNING INCORPORATED, SP-TI-3-1, CORNING, NY, 14831, US

(21) Appl. No.: 12/008,309

(22) Filed: Jan. 10, 2008

(51) Int. Cl. G01N 33/53

(52) U.S. Cl. 435/7.21; 435/7.2; 435/7.23

(57) ABSTRACT

An apparatus and non-invasive method to measure cell-cell interactions, such as T-cell and stem cell interactions, under physiologically relevant conditions, and optionally measure the effects of stimuli on the cell-cell interactions, as defined herein.

The entire disclosure of any publication, patent, or patent document mentioned herein is incorporated by reference.

### BACKGROUND

The disclosure is related to optical biosensors, particularly resonant waveguide grating (RWG) sensors, and the use of optical biosensors for probing cell-cell interactions, such as direct and indirect cell-cell communication, and to screening methods for substances that can modulate cell-cell interaction phenomena.

### SUMMARY

The disclosure provides an optical biosensor and conditions of culturing adherent cells onto the surface of the biosensor to form an adherent cell layer. A second type of cells can be provided to the medium such that they directly or indirectly interact with the adherent cells in the absence or presence of a modulator. The method monitors optical outputs throughout the process in a continuous or discontinuous manner. The disclosure enables monitoring of cell-cell communication and its modulations under different fluidic environments, for example, static, plate movement, liquid movement, and like environments or conditions by using different system settings.

### BRIEF DESCRIPTION OF THE DRAWINGS

FIGS. 1A and 1B illustrate the principles of RWG biosensor for probing cell-cell communication, in embodiments of the disclosure.

FIGS. 2A to 2C illustrate the optical signatures of CHO-ICAM1 cells in response to the direct interaction with natural killer cells under three different operational conditions, in embodiments of the disclosure.

FIG. 3 illustrates the amplitudes of the N-DMR signal mediated by natural killer cells as a function of the number of killer cells, in embodiments of the disclosure.

FIGS. 4A to 4D illustrate the modulation profile of the natural killer cell-induced DMR signal of the target CHO-ICAM1 cells by different modulators at different doses, in embodiments of the disclosure.

FIGS. 5A to 5F illustrate the modulation profile of the natural killer cell-induced response of the target cell layer (D1 cells) by different modulators at different doses, in embodiments of the disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and set forth only some of the many possible embodiments for the claimed invention.

### DEFINITIONS

“Assay,” “assaying” or like terms refers to an analysis to determine, for example, the presence, absence, quantity, extent, kinetics, dynamics, or type of a target cell's optical response upon interaction with, for example, an effector cell. The target cell is a cell attached onto the surface of a biosensor. The effector cell is a cell that does not attach to the surface of a biosensor, but can interact with the targeted cell directly or indirectly. A direct interaction can be achieved through the binding of a cell surface receptor of the effector cell (such as a T cell, a B cell, a helper T cell, a transmitter producing cell, a natural killer cell, etc.) with a cell surface molecule of the target cell (such as an antigen-presenting cell, a cancerous cell, a tumorogenic cell, a normal cell, an infected cell, etc.). The transmitter-producing cells can include, but not limited to, an insulin-producing cell (e.g., islet cells), a mast cell, and a monocyte. Alternatively, the effector can also co-immobilized with the target cell on the sensor surface, when the effector cell is a transmitter-releasing cell. An indirect interaction can be achieved through the signaling of the target cell mediated by the released transmitters (such as adenosine, insulin, adenosine triphosphate, interleukins) from the effector cell in response to stimulation. “Assay” or like terms can also refer to an analysis to determine, for example, the presence, absence, quantity, extent, kinetics, dynamics, or type of a target cell's optical response upon interaction with, for example, an effector cell such as mentioned above, for example, upon pre-treatment, post-treatment, or like stimulation with an exogenous stimuli, such as a ligand candidate compound, a viral particle, or a pathogen. In the context of the assay “pre-treatment” and “post-treatment” refer, respectively, to before and after cell-cell interaction has been initiated.

“Attach,” “attachment,” “adhere,” “adhered,” “adherent,” “immobilized,” or like terms generally refer to immobilizing or fixing, for example, a surface modifier substance, a compatibilizer, a cell, a ligand candidate compound, and like entities of the disclosure, to a biosensor surface, such as by physical absorption, chemical bonding, and like processes, or combinations thereof. Particularly, “cell attachment,” “cell adhesion,” or like terms refer to the interacting or binding of cells to a surface, such as by culturing, or interacting with cell anchoring materials, compatibilizer (e.g., fibronectin, collagen, lamin, gelatin, polylysine, etc.), or both.

“Adherent cells” refers to a cell or a cell line or a cell system, such as a prokaryotic or eukaryotic cell, that remains associated with, immobilized on, or in certain contact with the outer surface of a substrate. Such type of cells after culturing can withstand or survive washing and medium exchanging process, a process that is prerequisite to many cell-based assays. “Weakly adherent cells” refers to a cell or a cell line or a cell system, such as a prokaryotic or eukaryotic cell, which weakly interacts, or associates or contacts with the surface of a substrate during cell culture. However, these types of cells, for example, human embryonic kidney (HEK) cells, tend to dissociate easily from the surface of a substrate by physically disturbing approaches such as washing or medium exchange. “Suspension cells” refers to a cell or a cell line that is preferably cultured in a medium wherein the cells do not attach or adhere to the surface of a substrate during the culture. However, these suspension cells can be attached to the surface of a biosensor through electrostatic interactions or covalent coupling. “Cell culture” or “cell culturing” refers to the process by which either prokaryotic or eukaryotic cells are grown under controlled conditions. “Cell culture” refers to the culturing of cells derived from multicellular eukaryotes, especially animal cells, and can also refer to the culturing of complex tissues and organs.

“Cell” or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including natural cells, genetically modified cells, synthetic cell constructs, cell model systems, and like artificial cellular systems.

“Cell system” or like term refers to a collection of more than one type of cells (or differentiated forms of a single type of cell), which interact with each other, thus performing a biological, physiological, or pathophysiological function. Such cell system includes an organ, a tissue, a stem cell, a differentiated hepatocyte cell, or like systems.

“Detect” or like terms refer to an ability of the apparatus and methods of the disclosure to discover or sense a stimulus-induced cellular response and to distinguish the sensed responses for distinct stimuli.

“Pathogen” or like terms refer to, for example, a virus, a bacterium, a prion, and like infectious entities, or combinations thereof.

“Stimulus,” “therapeutic candidate compound,” “therapeutic candidate,” “prophylactic candidate,” “prophylactic agent,” “ligand candidate,” or like terms, refer to a molecule or material, naturally occurring or synthetic, which is of interest for its potential to interact with a cell attached to the biosensor or to influence or modulate a cell-cell interaction. A therapeutic or prophylactic candidate can include, for example, a chemical compound, a biological molecule, a peptide, a protein, a biological sample, a drug candidate small molecule, a drug candidate biologic molecule, a drug candidate small molecule-biologic conjugate, or like materials or molecular entity, and combinations thereof, which can specifically bind to or interact with at least one of a cellular target or a pathogen target such as a protein, DNA, RNA, an ion, a lipid or like structure or component of a living cell.

“Biosensor,” or like terms, refer to a device for the detection of an analyte that combines a biological component with a physicochemical detector component. The biosensor typically consists of three parts: a biological component or element (such as tissue, microorganism, pathogen, cells, or combinations thereof, such as cell-cell interactions), a detector element (operating, e.g., in a physicochemical way such as optical, piezoelectric, electrochemical, thermometric, or magnetic), and a transducer associated with both components. The biological component or element can be, for example, a live cell, a pathogen, or combinations thereof. In embodiments, an optical biosensor can comprise an optical transducer for converting a molecular recognition or molecular stimulation event in a living cell, a pathogen, or combinations thereof into a quantifiable signal.

An antigen or immunogen is a molecule that sometimes stimulates an immune response. Antigens are usually proteins or polysaccharides. Cells present their antigens to the immune system via a histocompatibility molecule. Depending on the antigen presented and the type of the histocompatibility molecule, several types of immune cells can become activated. Antigens can be classified in order of their origins, including exogenous antigens, endogenous antigens, autoantigens, and cancer antigens.

Exogenous antigens are antigens that have entered the body from the outside, for example by inhalation, ingestion, or injection. By endocytosis or phagocytosis, these antigens are taken into the antigen-presenting cells (APCs) and processed into fragments. APCs then present the fragments to T helper cells (CD4+) by the use of class II histocompatibility molecules on their surface. Some T cells are specific for the peptide, such as the MHC (major histocompatibility complex) complex. They become activated and start to secrete cytokines. Cytokines are substances that can activate cytotoxic T lymphocytes (CTL), antibody-secreting B cells, macrophages, and like particles.

Endogenous antigens are antigens that have been generated within the cell, as a result of normal cell metabolism, or because of viral or intracellular bacterial infection. The fragments are then presented on the cell surface in the complex with MHC class I molecules. If activated cytotoxic CD8+ T cells recognize them, the T cells begin to secrete different toxins that cause the lysis or apoptosis of the infected cell. In order to keep the cytotoxic cells from killing cells just for presenting self-proteins, self-reactive T cells are deleted from the repertoire as a result of tolerance (also known as negative selection which occurs in the thymus).

An autoantigen is usually a normal protein or complex of proteins (and sometimes DNA or RNA) that is recognized by the immune system of patients suffering from a specific autoimmune disease. These antigens should under normal conditions not be the target of the immune system, but due to mainly genetic and environmental factors the normal immunological tolerance for such an antigen has been lost in these patients.

Tumor antigens or Neoantigens are those antigens that are presented by MHC I or MHC II molecules on the surface of tumor cells. These antigens can sometimes be presented by tumor cells and never by the normal ones. In this case, they are called tumor-specific antigens (TSAs) and typically result from a tumor specific mutation. More common are antigens that are presented by tumor cells and normal cells, and they are called tumor-associated antigens (TAAs). Cytotoxic T lymphocytes that recognized these antigens may be able to destroy the tumor cells before they proliferate or metastasize. Tumor antigens can also be on the surface of the tumor in the form of, for example, a mutated receptor, in which case they will be recognized by B cells.

“Include,” “includes,” or like terms means including but not limited to.

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

“Consisting essentially of” in embodiments refers, for example, to a cell-cell surface composition, a method of making or using a cell-cell surface composition, formulation, or cell-cell composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, aberrant affinity of a stimulus for a cell surface receptor or for an intracellular receptor, anomalous or contrary cell activity in response to a ligand candidate or like stimulus, and like characteristics.

Thus, the claimed invention may suitably comprise, consist of, or consist essentially of: a method for characterizing a cell-cell interaction as defined herein, and a method for characterizing a cell-cell response to a stimulus as defined herein.

The indefinite article “a” or “an” and its corresponding definite article “the” as used herein means at least one, or one or more, unless specified otherwise.

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, and like abbreviations).

Specific and preferred values disclosed for components, ingredients, additives, cell-types, antibodies, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

In embodiments, the disclosure provides a method for characterizing a live cell-cell interaction, the method comprising:

providing a biosensor system having a target cell immobilized on the biosensor;

contacting the immobilized target cell with an effector cell; and

detecting the dynamic mass redistribution of the target cell with the biosensor.

In embodiments the method can further comprise, for example, contacting the target cell with a stimulus prior to contacting with the effector cell. The stimulus can be selected from, for example, a Golgi apparatus disrupter, a cell-cycle blocker, an apoptosis inducer, an actin polymerization inhibitor, a GTPase modulator, a phosphatidylinositol 3-kinase inhibitor, an apoptosis inhibitor, a cell surface receptor modulator, and like entities, or combinations thereof. The stimulus can be selected from, for example, brefeldin A, camptothecin, latrunculin A, Rac1 inhibitor, wortmannin, Kp7-6, and like entities, or combinations thereof.

In embodiments the method can further comprise, for example, contacting the target cell with a stimulus after contacting with the effector cell. The stimulus can be selected from, for example, a Golgi apparatus disrupter, a cell-cycle blocker, an apoptosis inducer, an actin polymerization inhibitor, a GTPase modulator, a phosphatidylinositol 3-kinase inhibitor, an apoptosis inhibitor, a cell surface receptor modulator, and like entities, or combinations thereof. The stimulus can be selected from, for example, brefeldin A, camptothecin, latrunculin A, Rac1 inhibitor, wortmannin, Kp7-6, and like entities, or combinations thereof.

The target cell can be, for example, at least one of a tumor cell, an infected cell, a normal cell, a stem cell, a cancerous cell, and like entities, or mixtures thereof. The effector cell can be, for example, at least one of a T cell, a helper T cell, a B cell, a transmitter-producing cell, and like entities, or mixtures thereof. The transmitter-producing cell can be, for example, at least one of an insulin-producing cell, an islet cell, a mast cell, a monocyte, and like entities, or mixtures thereof.

In embodiments, the target cell immobilized on the biosensor's surface can have, for example, a confluency of from about 0.5% to about 100%, including intermediate confluency values, and any ranges thereof.

In embodiments, the biosensor system can be, for example, a swept wavelength optical interrogation imaging system for a resonant waveguide grating biosensor, an angular interrogation system for a resonant waveguide grating biosensor, a spatially scanned wavelength interrogation system, surface plasmon resonance system, surface plasmon resonance imaging, and like biosensor systems, or a combination thereof. The swept wavelength optical interrogation imaging system can monitor a cellular response, for example, at single-cell resolution (see for example copending U.S. Provisional Application Ser. No. 60/997,908, filed Oct. 6, 2007, entitled “Single-Cell Label-Free Assay” assigned to Corning Inc.). The dynamic mass redistribution signal can be, for example, an optical signal that measures real-time kinetics of an effector cell contact-induced cellular response as a function of time. The dynamic mass redistribution signal can be, for example, an optical signal that measures an endpoint or multiple points of an effector cell contact-induced cellular response over time and throughout an effector cell contact-induced event. The biosensor imaging system can provide biosensor output that can include, for example, at least one of: the overall dynamics, the phase, the amplitude and kinetics of the phase, the transition time from one phase to another of the dynamic mass redistribution signal, or a combination thereof.

In embodiments, the disclosure provides a method for characterizing a live cell-cell response to a stimulus, the method comprising:

providing a biosensor imaging system having a target cell immobilized on the biosensor;

contacting the immobilized target cell with a first stimulus;

contacting the first stimulus-contacted immobilized target cell with an effector cell;

detecting the dynamic mass redistribution of the target cell with the biosensor; and

determining the cell-signaling difference effect of contacting the stimulus-contacted target cell with the effector cell.

The method can further comprise, for example, contacting the first stimulus-contacted immobilized target cell and the effector cell with a second stimulus. The second stimulus can be, for example, the same as the first stimulus or the second stimulus can be different from the first stimulus.

The following references and sources mention methods for studying cell-cell communications: Choudhuri, et al., 2005, “T-cell receptor triggering is critically dependent on the dimensions of its peptide-MHC ligand,” Nature, 436, 578-582; Grakoui, et al., 1999, “Immunological synapse: a molecular machine controlling T-cell activation,” Science, 285, 221-227; Glamann, et al., 2006, “Dynamic detection of natural killer cell-mediated cytotoxicity and cell adhesion by electrical impedance measurements,” Assay and Drug Dev. Tech., 4, 555-563; and http://stemcells.nih.gov.

Published studies of T-cell assays have apparently not been conducted under physiological relevant conditions, and commonly use, for example, fluorescence and cell fixation technologies. Such methods typically are invasive and require manipulations of cells (e.g., fixation, staining with fluorescently labeled antibodies, or both) and which manipulations can distort or interfere with the observed results.

In embodiments the disclosure provides non-invasive methods to measure cell-cell interactions or cell-cell communication processes under physiologically relevant conditions. Optical sensors including resonant waveguide grating (RWG) sensors can be used to measure or monitor the cell-cell interactions. As an example, RWG sensors were used to study the interaction of natural killer cells with adherent target cells. The natural killer (NK) cells can be used as effector cells to interact with target cells, see for example, D1-pluripotential bone marrow cell line (U.S. Pat. No. 6,082,364); and such interaction leads to the removal of apoptotic target cells. Significant loss in local mass within the bottom portion (i.e., with respect to the surface of the biosensor closest to the cell surface) of adherent target cells was observed and can be modulated by several types of modulators.

In embodiments the disclosure can provide a number of advantages over existing methodologies for measuring cell-cell interactions and consequences thereof, including for example: label-free detection where, e.g., an integrated signal arising from cell-cell interaction can be detected with optical biosensors by measuring the changes in the wavelength, interrogation angle, refractive index, or a combination thereof; high throughput assays are enabled by, e.g., simultaneous monitoring of 384 samples in a 384-well plate; physiologically relevant assay conditions can be used, e.g., in assays having whole live-cells in a cell culture medium; and simple assay processes, e.g., after target cells are confluently grown at the bottom of an Epic® sensor plate, effector cells (i.e., a second cell-type or line) can be added. One can then measure, for example, the kinetic interaction or the end-point readouts using optical biosensors such as with using an Epic® instrument (www.corning.com). The disclosed methods can significantly reduce or eliminate manual processing and create opportunities for more productive experimental design and data interrogation particularly in the cell biology and cell-cell interaction space.

### Cell-Cell Communication

Many cells in the human body work collaboratively with each other, rather than independently. For the cells to work together they interact or communicate with each other by sending or receiving signals. Cell-cell communication in organisms is associated with the accumulation of signal molecules that regulate gene expression, cellular function, or both. These signal molecules act at the systems level and, consequently, a wide variety of cellular functions are influenced. Cell-cell communication is essential throughout the life of the organism. Indeed, many diseases such as cancers are due in part to failures of normal cell-cell communication. Communication between a premalignant cell and neighboring cells can play a significant role in the development of full-fledged malignancy. Cell-cell communication is also essential for the proper development and regulation of functional tissues and organ systems.

### T-Cell-Cell Interactions

T cells belong to a group of white blood cells known as lymphocytes. T-cells are central players of the adaptive immune response, which help protect a host against different pathogens such as bacteria, fungi, viruses, and like entities. To perform effectively T-cells need to be activated. The activation process can lead to a variety of responses, such as proliferation, migration, cytokine production, apoptosis, and like responses. T-cell activation and recognition involve multiple steps and events with its target cells. The response by T-cells to activate or not is pivotal. An inappropriate or incorrect response can lead to an autoimmune disease while a failure to respond could lead to infection and death. To perform such a complex and sensitive task, a T-cell must respond to internal environmental cues that stimulate a complex signaling cascade. Antigen recognition by T-cells involves simultaneous interactions between many T-cell surface receptors, including for example, T-Cell Receptors (TCR), its coreceptors CD4 and CD8, the co-stimulatory receptors CD28 and CTLA-4, and the accessory molecule CD2 and their ligands such as the major histocompatibility complex (MHC) protein class I/II, pMHC, CD58, ICAM-1 on antigen presenting cells (APCs) or target cells.

T-cell antigen recognition is possibly the best understood cell-cell recognition process. Activation of T-cells by antigen presenting cells depends on the complex integration of signals that are delivered by multiple antigen receptors. Most receptor-proximal activation events in T-cells have been identified using multivalent anti-receptor antibodies, but not the more complex APC cells. The central event in the T-cell activation is the interaction of TCR with the antigenic peptide presented by the MHC of the antigen-presenting cell. Because the number of agonist peptide-MHC complexes can be very low (10-100 per APC) and the TCR are continuously modulated from the T-cell surface, sustained T-cell activation may likely require signal amplification through co-stimulatory molecules on the T-cell. This could present a significant challenge for the maintenance of a good period of T-cell activation in vitro. T-cell activation and recognition involve multiple steps and events with its target cells. A T-cell is only able to recognize a small group of related antigens and is not effective against many others. T-cells are suspension cells, which are mobile cells optimized for migration, receptor scanning, and signaling.

Apoptosis is a significant process in human disease and disease management. Failure to regulate apoptosis is common in several diseases such as AIDS (acquired immune deficiency syndrome or acquired immunodeficiency syndrome), autoimmune disorders, cancer, and neurodegenerative diseases. For reasons that are unclear, some cell lines (particularly leukemia cell lines) undergo apoptosis within 3 to 6 hours, whereas other cell lines (fibroblasts and solid tumor cell lines) take several days even though the same fraction of cells will ultimately die, see Kaufmann, S. H., et al., 2003, “Unit 18.2 Analysis of caspase activation during apoptosis,” Curr. Protocol in Cell Biol. (New York, John Wiley and Sons, Inc.).

### Clusters of Differentiation Antigens

The analysis of leukocyte cell surface molecules has provided a more complete understanding of immunological phenomena. The identification of these molecules began with the characterization of allotypic differences among inbred mouse strains, and was further transformed by the advent of monoclonal antibody technology. Approximately 350 clusters of differentiation (CD) antigens have been identified on leukocytes (http://www.sciencegateway.org/resources/prow/index.html). The human leukocyte antigen system (HLA) is the name of the human major histocompatibility complex (MHC). This group of genes resides on chromosome 6, and encodes cell-surface antigen-presenting proteins and many other genes. The major HLA antigens are essential elements in immune function. They also have a role in disease defense such as reproduction, and cancer.

The cluster of differentiation (CD) is a protocol used for the identification and investigation of cell surface molecules present on leukocytes. CD molecules can act in numerous ways, often acting as receptors or ligands (the molecule that activates a receptor) important to the cell. A signal cascade is usually initiated, altering the behavior of the cell. Some CD proteins do not play a role in cell signaling, but have other functions, such as cell adhesion.

The CD system is commonly used as cell markers; this allows cells to be defined based on what molecules are present on their surface. These markers are often used to associate cells with certain immune functions or properties. While using one CD molecule to define populations is uncommon (though a few examples exist), combining markers has allowed for cell types with very specific definitions within the immune system.

CD molecules are utilized in cell sorting using various methods including flow cytometry. Cell populations are usually defined using a ‘+’ or a ‘−’ symbol to indicate whether a certain cell fraction expresses or lacks a CD molecule. For example, a “CD34+, CD31−” cell is one that expresses CD34, but not CD31. This CD combination typically corresponds to a stem cell, opposed to a fully differentiated endothelial cell.

Two commonly used CD molecules are CD4 and CD8, which are generally used as markers for helper and cytotoxic T cells, respectively. When defining T cells, these molecules are defined in combination with CD3+, as some other leukocytes also express these CD molecules (some macrophages express low levels of CD4, dendritic cells express high levels of CD8). CD4 is also an essential receptor during HIV infection, allowing the HIV to bind to the helper T cell and destruction of CD4+ T cells. The relative abundance of CD4+ and CD8+ T cells is often used to monitor the progression of an HIV infection.

### Protein-Protein Interaction Related to T-Cell Activations

In living cells, membrane receptors transduce ligand binding into signals that initiate, for example, proliferation, specialization, and secretion of signaling molecules. Spatial organization of such receptors regulates signaling in several key immune cell interactions. In the most extensively studied of these, a T-cell recognizes membrane-bound antigen presented by a target cell, and forms a complex junction called the immunological synapse (IS). The importance of spatial organization at the IS and the quantification of its effect on signaling remain controversial topics. Researchers have successfully investigated the immunological synapse using lipid bilayers supported on solid substrates as model antigen-presenting membranes. Recent technical developments have enabled micron- and nanometer-scale patterning of supported lipid bilayers and have been applied to immune cell studies with intriguing results, including spatial mutation of the immunological synapse. For example, one traditional way to study molecule interaction with T-cells is to use a fluorescence labeled antibody or a green fluorescent protein (GFP) tagged antigen (e.g., CD3, Zap70) after contacting the T-cells with an artificial lipid bilayer containing MHC class II and ICAM-1 (see e.g., Mossmanabc, K., et al., “Micropatterned supported membranes as tools for quantitative studies of the immunological synapse,” Chem. Soc. Rev., 2007, 36, 46-54). Protein-protein interactions have been studied with, for example, Western hybridization, surface plasmon resonance, or flow cytometry.

### T-cell and Antigen Presenting Cells Interaction

The specialized junction between a T lymphocyte and an antigen-presenting cell, the immunological synapse, consists of a central cluster of T-cell receptors surrounded by a ring of adhesion molecules. Immunological synapse formation has been shown in embodiments of the present disclosure to be an active and dynamic process that allows T-cells to distinguish potential antigenic ligands. Initially, T-cell receptor ligands were engaged in an outermost ring of the nascent synapse. Transport of these complexes into the central cluster was dependent on T-cell receptor-ligand interaction kinetics. Finally, formation of a stable central cluster at the heart of the synapse was a determinative event for T-cell proliferation. The dynamic interactions between T-cells and antigen-presenting cells can be probed, for example, with fluorescence photobleaching recovery using engineered antigen-presenting cells and T-cells. At given time intervals cells are fixed and stained. The staining pattern can be then examined using confocal microscopy or a digital imaging system. Alternatively, the T-cell-APC interaction can be assayed using electrical impedance measurements.

Interactions with Stem Cells

Stem cells have the remarkable potential to develop into many different cell-types in the body. Serving as a sort of repair system for the body, they can theoretically divide without limit to replenish other cells as long as the person or animal is still alive. When a stem cell divides, each new cell has the potential to either remain a stem cell or become another type of cell with a more specialized function, such as a muscle cell, a red blood cell, or a brain cell. Stem cells have two important characteristics that distinguish them from other types of cells. First, they are unspecialized cells that renew themselves for long periods through cell division. The second is that under certain physiologic or experimental conditions, they can be induced to become cells having special or differentiated functions, such as the beating cells of the heart muscle or the insulin-producing cells of the pancreas.

Scientists primarily work with two kinds of stem cells derived from animals or humans: embryonic stem cells and adult stem cells. Each has different functions and characteristics. In some adult tissues, such as bone marrow, muscle, and brain, discrete populations of adult stem cells generate replacements for cells that are lost through normal wear and tear, injury, or disease. Scientists desire to further study stem cells in the laboratory so they can learn about their essential properties and what makes them different from specialized cell-types. As scientists learn more about stem cells, it may become possible to use the cells not only in cell-based therapies, but also for example, in screening new drugs and toxins, and understanding birth defects, and like conditions or applications.

Scientists have recently and unexpectedly found adult stem cells in many more tissues than once thought possible. This finding has led scientists to ask whether adult stem cells could be used further for transplants. Adult blood forming stem cells from bone marrow have been used in transplants for over 30 years. Certain kinds of adult stem cells seem to have the ability to differentiate into a number of different cell-types, given the right conditions. If this differentiation of adult stem cells can be controlled in the laboratory, these cells may become the basis of therapies for many serious common diseases.

Research on adult stem cells began about 40 years ago. In the 1960s, researchers discovered that the bone marrow contains at least two kinds of stem cells. One population, called hematopoietic stem cells, forms all blood cell-types in the body. A second population, called bone marrow stromal cells, was discovered shortly thereafter. A potential advantage of using stem cells from an adult is that the patient's own cells could be expanded in vitro and then reintroduced into the patient. The use of the patient's own adult stem cells would mean that the cells would be less likely to be rejected by the immune system. This aspect represents a significant advantage as immune rejection is a difficult problem that has only been circumvented with immunosuppressive drugs.

Scientists are searching for ways to grow adult stem cells in cell culture and manipulate them to generate specific cell-types so they can be used as a cell source to treat injury or disease. Scientists do not agree on the criteria that should be used to identify and test adult stem cells. However, one or more of the following three methods are often used: 1) labeling the cells in a living tissue with molecular markers and then determining the specialized cell-types they generate; 2) removing the cells from a living animal, labeling them in cell culture, and transplanting them back into another animal to determine whether the cells repopulate the tissue of origin; and 3) isolating the cells, growing them in cell culture, and manipulating them, often by adding growth factors or introducing new genes, to determine what differentiated cells types they can become.

### Interactions Between a Natural Killer Cell and its Target Cell

Natural killer cells (or NK cells) are a type of cytotoxic lymphocyte which constitutes a major component of the innate immune system. NK cells play a major role in the rejection of tumors and cells infected by viruses. The cells kill by releasing small cytoplasmic granules of proteins called perforin and granzyme that cause the target cell to die by apoptosis. Upon release in close proximity to a cell slated for killing, perforin forms pores in the cell membrane of the target cell through which the granzymes and associated molecules can enter, inducing apoptosis.

NK-cells are defined as large granular lymphocytes that do not express T-cell antigen receptors (TCR) or Pan T marker CD3 or surface immunoglobulins (Ig) B cell receptor but which usually express the surface markers CD16 (FcγRIII) and CD56 in humans, and NK1.1/NK1.2 in certain strains of mice. They were named “natural killers” because of the initial notion that they do not require activation in order to kill cells that are “missing self” markers of major histocompatibility complex (MHC) class I.

NK cells are activated in response to interferons or macrophage-derived cytokines. They serve to contain viral infections while the adaptive immune response is generating antigen-specific cytotoxic T cells that can clear the infection. Patients deficient in NK cells prove to be highly susceptible to early phases of herpes virus infection. In order for NK cells to defend the body against viruses and other pathogens, they require mechanisms which enable the determination of whether a cell is infected or not. To control their cytotoxic activity, NK cells possess two types of surface receptors: “activating receptors” and “inhibitory receptors”. Most of these receptors are not unique to NK cells and can be present in other T cell subsets as well.

These inhibitory receptors recognize MHC class I alleles, which could explain why NK cells kill cells possessing low levels of MHC class I molecules. This inhibition is crucial to the role played by NK cells. MHC class I molecules consist of the main mechanism by which cells display viral or tumor antigens to cytotoxic T-cells. A common evolutionary adaptation to this, seen in both intracellular microbes and tumors, is a chronic down-regulation of these MHC I molecules, rendering the cell impervious to T-cell mediated immunity. It is believed that NK cells, in turn, evolved as an evolutionary response to this adaptation, as the loss of the MHC would deprive these cells of the inhibitory effect of MHC and render these cells vulnerable to NK-cell mediated lysis.

### Optical Biosensor Systems

The disclosure provides methods to study cell-cell communication and also its modulation by compounds under different operational environments, such as static conditions and continuous fluidic movement generated by mechanical movement of, for example, cell assay microplates which the target cells are adhered to. The cell-cell communication, particularly the T-cell and antigen-presenting cell interaction, is sensitive to the fluidic environment. Any fluidic movement, as commonly encountered, for example, in the human body, may interfere with the interaction of T-cells interacting with their target cells. The cell-cell interaction under static condition (i.e., no moving parts or no fluidic movement during the assay, except for the step wherein a second solution containing the effector cells is introduced) can be assayed, for example, using an angular interrogation system, as described in the optical interrogation system and method for 2-D sensor arrays (see PCT Patent Appl. No. WO2006107967 A1, assigned to Corning Inc.), and a wavelength swapping system, as described in the label-free high throughput biomolecular screening system and method (see PCT Patent Appl. No. WO2007018872 A1, assigned to Corning Inc.). The cell-cell interaction under continuous fluidic movement can be assayed using the wavelength interrogation system, as described in the spatially scanned optical reader system and method for using same (see U.S. Publication No. US20060205058 A1, assigned to Corning Inc.). In this wavelength interrogation system, a series of illumination/detection heads are arranged in a linear fashion, so that reflection spectra are collected from a subset of wells within the same column of a 384-well microplate at the same time. The whole plate can be scanned by the illumination/detection heads so that each sensor can be addressed multiple times, and each column can be addressed in sequence. Thus, the biosensor microplates having the target cells are continuously scanned across an array of optical fibers; such scanning process can lead to certain continuous fluidic movement. This disclosed system offers flexibility in, for example, scanning such that the scanning can be continuous or discontinuous depending on the assay format selected.

### Theory of Operation

The Corning® Epic® cell assay system use resonant waveguide grating (RWG) sensors to monitor in real-time the stimulus-induced dynamic mass redistribution within the bottom 200 nm portion of adherent target cells.

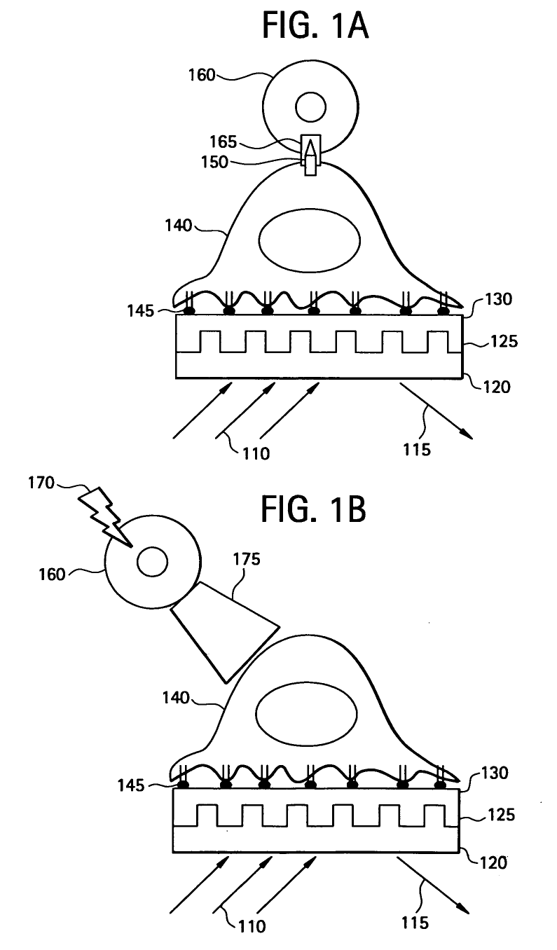

Referring to the Figures, FIG. 1 illustrates the principles of RWG biosensor for probing cell-cell communication. The biosensor consists of a substrate (120), a waveguide thin film wherein a periodic grating structure is embedded (125), and optionally a surface chemistry (130). The target cell (140) is attached onto the surface of the biosensor, preferably through extracellular adhesion complexes (145). Alternatively, the target cell can be attached onto the surface of the biosensor, through electrostatic interactions or covalent coupling (U.S. Provisional Patent Application, 60/904,129, assigned to Corning Inc., filed Feb. 28, 2007, entitled “Surfaces and Methods for Biosensor Cellular Assays”). The biosensor uses a light source to illuminate (110) the biosensor, such that only the resonant light (115) is reflected back and detected whose optical contents (i.e., wavelength/angle, peak width at half maximum, etc) are used as optical outputs for analyzing cell-cell interactions and their modulations by a compound. For example, target cells were grown to cover the bottom of the Epic® 384-well plate wells. Then T-cells such as the natural killer cells were dispensed on top of the target cells in each well with a 384-channel liquid handler. The added T-cells are situated beyond the sensor detecting zone since they reside substantially on the top of or above target cells, which are approximately several microns in height. T-cells are highly mobile cells adept at, for example, migration, receptor scanning, and signaling. T-cell activation and recognition involve multiple steps and events with its target cells. Modulation experiments of cell-cell interaction can be used to produce significant information regarding, for example, the influence of a pharmaceutical candidate compound on the control cell-cell interaction. The integrated response signal can be recorded with the Epic® optical biosensor instrument and analyzed.

In embodiments, a target cell (140) can be immobilized onto the surface (130) of a biosensor, such as resonant waveguide grating biosensor in which a waveguide thin film having a periodic grating structure (125) has been deposited or fused onto a substrate (e.g., glass or plastic) (120), such as through extracellular adhesion complexes (145). A light source (110) can be used to illuminate the biosensor, such that only the resonant light (115), having optical content (i.e., wavelength, angle, or shape) which is a measure of cell-cell interaction and its modulation, that can be back-reflected and detected.

In embodiments, the cell-cell interaction can be achieved, for example, by the recognition and binding of an effector cell (160) to the target cell (140), such as the direct interaction of a receptor (165) presented in the effector cell with an antigen (150) presented in the target cell. Such direct interaction mediates signaling in the target cells, which can be non-invasively monitored by the biosensor.

In embodiments as shown in FIG. 1B, the cell-cell interaction can be achieved through indirect interaction between the effector cell (160) and the target cell (140). Instead of direct binding between the cell surface markers or molecules of both the effector cell and the target cell, a compound or stimulus (170) can trigger the release of transmitters or molecules from the effector cells through exocytosis (175), and the released entities can result in signaling of the target cells. In many instances, the cell-cell interaction, such as T cell and its target cell interaction involves both types of interactions. In this instance, the effector cell can be either suspended in solution, or immobilized on the sensor surface such that it is physically in close proximity to the target cell.

### Optical Biosensors for Monitoring Natural Killer Cell-Induced Apoptosis of Antigen-Presenting Cells (CHO-ICAM1)

Adhesion molecules participate in many stages of immune response. Cell adhesion molecules form a large group of proteins that perform several functions. A majority of adhesion molecules can be grouped into, e.g., integrins, cadherins, selecting, and immunoglobulin superfamilies. Modulation of cell adhesion can be significant in, for example, inhibition of tumor metastasis, suppression of the immune response in autoimmune diseases, and for improving drug delivery through biological barriers. Blocking the adhesion molecular interaction or modulation of adhesion molecules consequently produces biological effects of therapeutic value, for example, making adhesion molecules attractive candidates for drug-design.

Intercellular adhesion molecules (ICAMs) are molecules that promote adhesion between cells. Examples include adhesion from most white blood cells, related to their immunological response to wound or bacterial infection. Chinese hamster ovary (CHO) cells stably expressing ICAM subtype 1 (CHO-ICAM1) can be used as the target cells. The CHO-ICAM1 cells express high levels of human ICAM-1. The CHO-ICAM1 cells were directly cultured onto the surface of Corning® Epics 96-well or 384-well biosensor microplate to form a confluent monolayer. The natural killer cells, NK cell line NK92, were used as the effector cells.

It is known that NK92 cells can recognize ICAM1 presented in the target cells, resulting in apoptosis of the target cells. When the target cells become apoptotic, there was observed a significant loss in local mass within the target cell layer induced be the detachment or release of cellular materials by the apoptotic cells.

In embodiments, the cell cultures can be, for example, adherent cells or suspension cells, depending on their conditions for adherency to achieve appropriate growth. Consequently, appropriate surface chemistries and culture conditions may need to be selected. The cell cultures can also be, for example, transformed cell lines, immortalized cells, primary cells, stem cells, tissue, or like cell cultures.

The imaging biosensor can be, for example, an SPR imaging system, an ellipsometry imaging system, a swept wavelength optical interrogation imaging system, or like imaging system.

### EXAMPLES

The following examples serve to more fully describe the manner of using the disclosure, as well as to further illustrate and demonstrate specific examples of best modes contemplated for carrying out various aspects of the disclosure. These examples do not limit the scope of the disclosure, but rather are presented for illustrative purposes.

### Example 1

### NK92-Induced DMR Signal of CHO-ICAM1 Cells is Sensitive to the Fluidic Movement

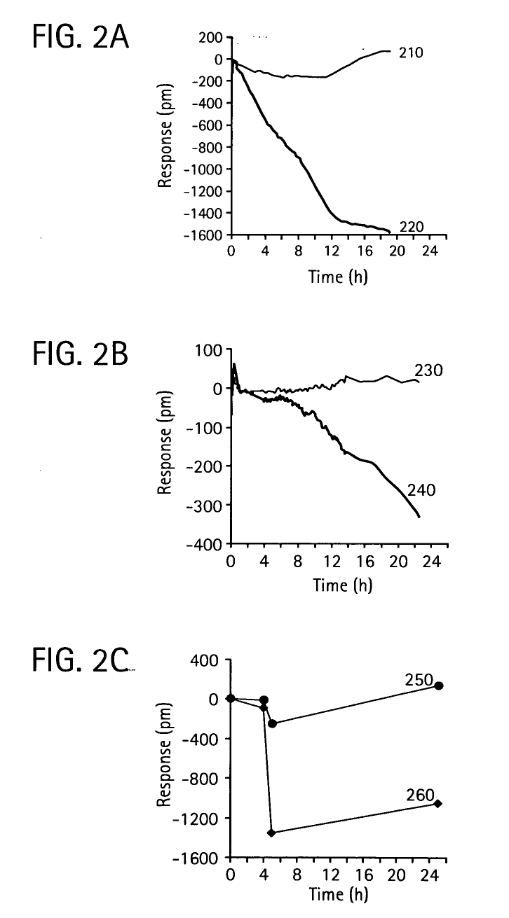

FIG. 2 illustrate the optical signatures of CHO-ICAM1 cells in response to the interaction with natural killer cells under three different operational conditions. FIG. 2A is the DMR (Dynamic Mass Redistribution) signal obtained using a static optical system (e.g., angular interrogation system). FIG. 2B is the DMR signal obtained using a scanning optical system (e.g., Corning® Epic® system), where the microplate having a sensor in each well is scanned across an array of optics in a continuous fashion. FIG. 2C is the DMR signal obtained using discontinuous end-point measurements with the scanning optical system.

As shown in FIG. 2, NK92 cells triggered a unique DMR signal of the target CHO-ICAM1 cell layer. The DMR signal is characterized by a prolonged decrease in the resonant wavelength or angle, meaning a loss in local mass density within the sensor detection zone, as defined by the exponential decaying nature of the evanescent wave (about 150 nanometers). Such a signal is termed as Negative-DMR (N-DMR). The kinetics and amplitude of the NK92 cell-induced N-DMR were found to be dependent on the experimental conditions, including, for example, static, continuous scanning, and discontinuous scanning. Using the static angular interrogation system, the N-DMR event proceeds with the fastest pace and reach the greatest maximum decrease (220) (e.g., about 1,400 pm shift in the resonant wavelength), corresponding to about 50% loss in the mass of CHO-ICAM1 cell layer within the detection zone of the biosensor (compared to control, where for example, the target cell is the parental cell line, such as Chinese hamster ovary cell, which does not express ICAM1 molecules) (210)). Such assay condition-dependency is expected. The NK92 cells can recognize the ICAM1 presented in the target cells, and trigger the apoptosis of the target cells. Under static conditions, the NK cells can remain in a location sufficiently long to have a sustained impact on the target cells over an extended time period, thus leading to faster kinetics and higher percentage of cell apoptosis. The discontinuous end-point measurements mimic the results obtained under the static conditions, since it only involves minimal plate movement (FIG. 2C, 260 v. 250). Conversely, under continuous scanning conditions, the relatively small turbulence of fluid (i.e., the medium covering the target cells) generated by the plate movement can possibly lead to the on-and-off interaction of the NK92 cells with the ICAM1 presented on the target cell surface (FIG. 2B, 240 v. 230). Both DMR responses (230 and 250) were obtained using CHO cells as the target cell, which was used as a negative control.

### NK92-Induced DMR Signal of CHO-ICAM1 Cells is Dependent on the Ratio of Killer Cells to the Target Cells

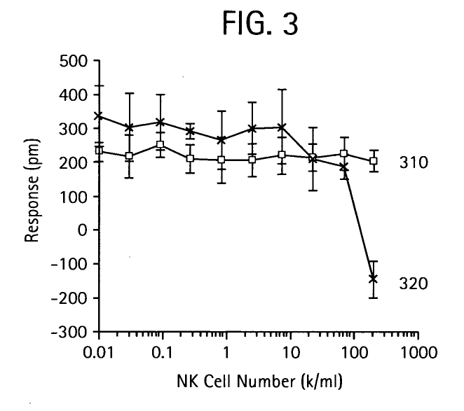

FIG. 3 illustrates the amplitudes of the N-DMR signal mediated by natural killer cells as a function of the number of killer cells. Two types of target cells were compared: Chinese hamster ovary (CHO cells) (310); and engineered CHO cells having stably expressed ICAM1 antigens (320). Both types of cells were cultured onto the surface of Epic® 384-well biosensor microplates until they reached high confluency (about 100%). The natural killer cells suspended in the medium solution were introduced in each well, and the optical responses were recorded using the spatially scanned wavelength interrogation system using the discontinuous mode. Each data point represented an average of 16 replicates.

As shown in FIG. 3, the NK92 cells at all doses examined did not trigger significant loss in local mass within the CHO-K1 cells. The CHO-K1 is the parental cell line of CHO-ICAM1, and doses not express ICAM1. Conversely, the NK92 cells resulted in significant N-DMR signal (310), whose amplitude was dependent on the ratio of NK92 cells to the CHO-ICAM1 cells (320). Those results suggest that ICAM1 is crucial for the recognition of the target cells by NK92 cells. In addition, DNA gel electrophoresis analysis showed that the treatment of CHO-ICAM1 with NK92 cells led to DNA fragmentation, a characteristic of cell apoptosis (data not shown). These results are consistent with literature that NK92 indeed can recognize and subsequently cause the death of ICAM1-presenting cells. It is interesting to note that the present assay leads to higher sensitivity, compared to the conventional methods. It has been reported that using conventional assay technologies, the NK92-mediated cytotoxicity and apoptosis is typically detectable at an Effector/Target (E/T) ratio larger than 1 (Sun, et al., Cell Res., 2004, 14, 67). Using the present biosensor-based assays, there is notable effects on ICAM1 cells when the NK cell number is at 200 k/mL, which is equivalent to E/T ratio of 0.5.

### NK92-Induced DMR Signal of CHO-ICAM1 Cells can be Modulated by Pharmacological Agents

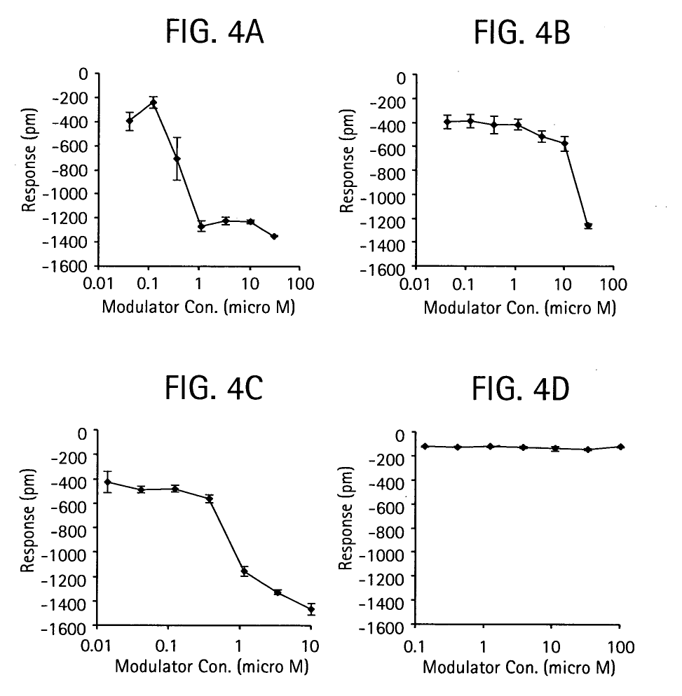

The impacts of four pharmacological agents were examined on the NK92 cell-induced DMR signal of CHO-ICAM1 cells. FIG. 4 illustrate the modulation profile (response v. modulator concentration in microM) of the natural killer cell-induced DMR signal of the target cell layer by four different modulators at different doses:

FIG. 4A brefeldin; FIG. 4B camptothecin; FIG. 4C vinblastine; and FIG. 4D caspase 3 inhibitor.

Brefeldin A (BFA) is a fungal metabolite which disrupts the structure and function of the Golgi apparatus. It is an activator of the sphingomyelin cycle.

Camptothecin blocks the cell cycle in S-phase at low dose and induces apoptosis at high dose in a large number of normal and tumor cell lines by cell cycle-dependent and cell cycle-independent processes. It binds irreversibly to the DNA-topoisomerase I complex.

Vinblastine is a toxic compound that induces apoptosis in cultured hepatocytes and human lymphoma cells. It affects interaction of tubulin with microtubule-associated proteins, specifically Tau and MAP2 and depolymerizes microtubules. Caspase 3 inhibitor, a potent cell-permeable and irreversible caspase 3 inhibitor, blocks caspase-3 activity as well as caspase-6, caspase-7, caspase-8, and caspase-10 functions. The inhibitor suppresses apoptosis.

The pretreatment of CHO-ICAM1 cells with BFA, camptothecin, or vinblastine, dose dependently increases the NK92-induced N-DMR, suggesting that these agents can act synergistically with the NK92 cells to result in the apoptosis of the target CHO-ICAM1 cells. On the other hand, the presence of caspase 3 inhibitor has little effect on the NK92-induced N-DMR event of CHO-ICAM1 cells, suggesting that the NK92-induced apoptosis of CHO-ICAM1 involves different pathways, other than the caspase 3 pathway.

The pretreatment of CHO-ICAM1 cells with U0126 (a MEK1/2 inhibitor) dose-dependently suppressed the NK92-induced N-DMR signal (data not shown), suggesting that the NK92 induced cell apoptosis proceeds through the mitogen-activated protein kinase pathway.

Regents—Caspase 3 inhibitor was from BD Bioscience (San Jose, Calif.). Brefeldin A and U0126 were from Tocris (St. Louis, Mo.). Camptothecin and vinblastine were from Sigma Chemical Co. (St. Louis, Mo.). Corning® Epic® 96-well and 384-well microplates were obtained from Corning Incorporated (Corning, N.Y.), and cleaned by exposure to high intensity UV light (UVO-cleaner, Jelight Company Inc., Laguna Hills, Calif.) for 6 minutes before use.

Cell culture—Natural killer cells-NK92MI, both parental CHO-K1 and engineered CHO expressing ICAM1 receptor (CHO-ICAM1) were purchased from American Type Culture Collection (Manassas, Va.). NK92MI cells were grown in RPMI medium plus 12.5% horse serum and 12.5 fetal bovine serum (FBS) and antibiotics. CHO-K1 cells and CHO-ICAM1 cells were grown in RPMI supplemented with 10% fetal bovine serum (FBS) and antibiotics. Approximately 1-2×10⁴ target cells of CHO-K1 or CHO-ICAM1 in 50 μl medium were placed in each well of Epic® 384-well microplate. After cell seeding, the cells were cultured at 37° C. under air/5% CO₂ until a high degree, e.g. 90%, of confluence was reached (e.g., about 24 hrs). Cells were continuously cultured for over night with their corresponding media without any serum to keep cells at a quiescent state and cell confluence was over 95%.

Epic® system and DMR assays—An Epic® beta system was used during this study. For cell-cell interaction study, target cell density was at 95-100% confluence and the cells were finally maintained with the appropriate medium as described above. Cell number of NK92MI was counted with Beckman Coulter Particle Counter (Beckman Coulter, Fullerton, Calif.). Unless stated otherwise, a ratio of 1 between killer cells and target cells was used. NK92MI cells were suspended in RPMI plus 2% FBS. A 20-microliter solution of NK92MI was aliquoted to each well of a normal 384-well microplate (Corning Inc., Corning, N.Y.). The plates of the target-cell and the natural killer cell were incubated in the Epic® instrument for at least 30 minutes to reach 28° C. For pharmacological agent studies, the Icam1 cells had been pretreated with the agents for 1 hour before the NK cells were introduced. All studies were carried out at 28° C. with the lid of the microplate on except for a short period of time (e.g., about several seconds) when the solution was introduced, in order to minimize the effect of temperature fluctuation and evaporative cooling.

### Use of an Optical Biosensor for Monitoring the Natural Killer Cell-Induced Apoptosis of Stem Cells (Multipotential Bone Marrow Cell Line D1)

It is known that NK92 cells can recognize the target cells under certain conditions, such as apoptotic target cells, infected by virus or bacteria, or both conditions. When the target cells become apoptotic there is significant loss in local mass within the target cell layer induced be the detachment or release of cellular materials from the apoptotic cells.

### NK92-Induced Optical Signal of D1 Cells can be Modulated by Pharmacological Agents

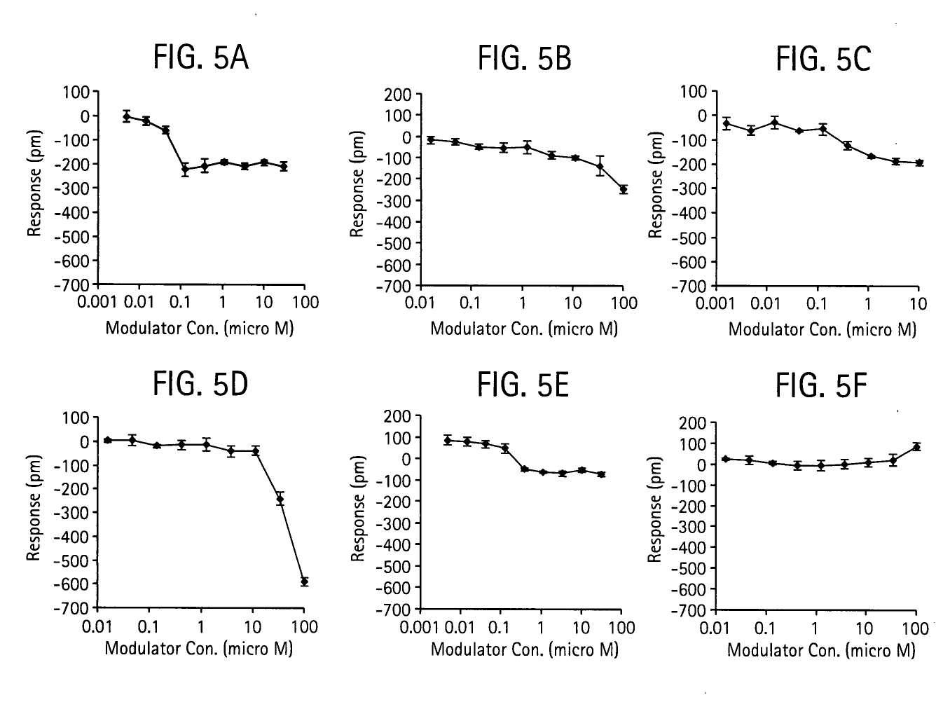

The impact of six pharmacological agents was examined on the NK92 cell-induced optical signal of D1 cells. FIG. 5 illustrate the modulation profile (response in picometers v. modulator concentration in micromolar) of the natural killer cell-induced response of the target cell layer by different pharmacological agents as modulators at different doses: FIG. 5A brefeldin A; FIG. 5B camptothecin; FIG. 5C latrunculin A; FIG. 5D Rac1 inhibitor; FIG. 5E wortmannin; and FIG. 5F Kp7-6. Brefeldin A (BFA), and Camptothecin are defined above. Kp7-6 is an exocyclic cystine-knot peptide that specifically antagonizes Fas/FasL-mediated cellular apoptotic signals, for example, it can reduce 58% of FasL-induced apoptosis in Jurkat cells at 1 mg/mL. Latrunculin inhibits actin polymerization in vitro and disrupts microfilament organization as well as microfilament-mediated processes. Rac1 inhibitor is a cell-permeable pyrimidine compound that specifically and reversibly inhibits Rac1 GDP/GTP exchange activity by interfering with Rac1 interaction with the Rac-specific GEF (guanine nucleotide exchange factor) Trio and Tiam 1 (IC₅₀ about 50 μM). Rac1 inhibitor effectively inhibits Rac 1-mediated cellular functions in NIH3T3 and PC-3 cells (effective dose about 50 to 100 μM). Rac1 inhibitor exhibits no effect on Cdc42 or RhoA activation, nor does it affect Rac1 interaction with BcrGAP or PAK1. Wortmannin is a potent, selective, cell-permeable and irreversible inhibitor of phosphatidylinositol 3-kinase.

The pretreatment of D1 cells with BFA, camptothecin, latrunculin A, Rac1 inhibitor, or wortmannin, dose dependently increases the NK92-induced signals, suggesting that these agents can act synergistically with the NK92 cells to result in the apoptosis of the target cells. Furthermore, Rac1 inhibitor significantly contributes to D1 cell interaction with NK cells and D1 cell apoptosis process. In contrast, the presence of apoptosis inhibitor Kp7-6 has dose-dependent protection on the NK92-induced response event of D1 cells, suggesting that the NK92-induced apoptosis of D1 is at least partially mediated through Fas/FasL pathway.

Regents Kp7-6 and Rac1 inhibitor were from BD Bioscience (San Jose, Calif.). Brefeldin A and wortmannin were from Tocris (St. Louis, Mo.). Camptothecin and latrunculin A were from Sigma Chemical Co. (St. Louis, Mo.). Corning® Epic® 384-well microplates were from Corning Inc. (Corning, N.Y.), and cleaned by exposure to high intensity UV light (UVO-cleaner, Jelight Company Inc., Laguna Hills, Calif.) for 6 minutes before use.

Cell culture—Natural killer cells-NK92MI and the multipotent mouse bone marrow D1 cell line were from American Type Culture Collection (Manassas, Va.). NK92MI cells were grown in RPMI medium plus 12.5% horse serum and 12.5 fetal bovine serum (FBS) and antibiotics. D1 cells were grown in Dulbecco's Modified Eagle's Medium supplemented with 10% fetal bovine serum (FBS) and antibiotics. Approximately 1×10⁴ to 2×10⁴ target cells of D1 in 50 μL medium were placed in each well of Epic® 384-well microplate. After cell seeding, the cells were cultured at 37° C. under air/5% CO₂ until a high degree, e.g., 90% of confluence was reached (about 24 hrs). Cells were continuously cultured overnight in their corresponding media without any serum to keep cells at a quiescent state and cell confluence was over about 95%.

Epic® system and DMR assays—Epic® beta system was used during this study. For cell-cell interaction study, target cell density was at about 95-100% confluence and the cells were finally maintained with the appropriate medium as described above. Cell number of NK92MI was counted with Beckman Coulter Particle Counter. Unless stated otherwise, a ratio of 1 between killer cells and target cells was used. NK92MI cells were suspended in RPMI plus 2% FBS. A 20 μL aliquot of NK92MI solution was added to each well of a normal 384-well microplate (Corning Inc., Corning, N.Y.). The plates of the target cells and the natural killer cells were incubated in the Epic® instrument for at least 30 minutes to reach 28° C. For pharmacological agent studies, the D1 cells were pretreated with the agents for 1 hour before NK cells were introduced. All studies were carried out at 28° C. for consistency and having the lid of the microplate continuously on except for a short period of time (e.g., about seconds) when the solution was introduced to minimize the effect of temperature fluctuation and evaporative cooling.

The disclosure has been described with reference to various specific embodiments and techniques. However, it should be understood that many variations and modifications are possible while remaining within the spirit and scope of the disclosure.

1. A method for characterizing a live cell-cell interaction, the method comprising:
providing a biosensor system having a target cell immobilized on the biosensor;
contacting the immobilized target cell with an effector cell; and
detecting the dynamic mass redistribution of the target cell with the biosensor

2. The method of claim 1 further comprising contacting the target cell with a stimulus prior to contacting with the effector cell.

3. The method of claim 2 wherein the stimulus is selected from the group consisting of a Golgi apparatus disrupter, a cell-cycle blocker, an apoptosis inducer, an actin polymerization inhibitor, a GTPase modulator, a phosphatidylinositol 3-kinase inhibitor, an apoptosis inhibitor, a cell surface receptor modulator, and combinations thereof.

4. The method of claim 2 wherein the stimulus is selected from the group consisting of brefeldin A, camptothecin, latrunculin A, Rac1 inhibitor, wortmannin, Kp7-6, and combinations thereof.

5. The method of claim 1 further comprising contacting the target cell with a stimulus after contacting with the effector cell.

6. The method of claim 5 wherein the stimulus is selected from the group consisting of a Golgi apparatus disrupter, a cell-cycle blocker, an apoptosis inducer, an actin polymerization inhibitor, a GTPase modulator, a phosphatidylinositol 3-kinase inhibitor, an apoptosis inhibitor, a cell surface receptor modulator, and combinations thereof.

7. The method of claim 5 wherein the stimulus is selected from the group consisting of brefeldin A, camptothecin, latrunculin A, Rac1 inhibitor, wortmannin, Kp7-6, or combinations thereof.

8. The method of claim 1 wherein the target cell comprises at least one of a tumor cell, an infected cell, a normal cell, a stem cell, a cancerous cell, or mixtures thereof.

9. The method of claim 1 wherein the effector cell comprises at least one of a T cell, a helper T cell, a B cell, a transmitter-producing cell, or mixtures thereof.

10. The method of claim 9 wherein the transmitter-producing cell comprises at least one of an insulin-producing cell, an islet cell, a mast cell, a monocyte, and mixtures thereof.

11. The method of claim 1 wherein the target cell immobilized on the biosensor's surface has a confluency of from about 0.5% to about 100%.

12. The method of claim 1 wherein the biosensor system comprises a swept wavelength optical interrogation imaging system for a resonant waveguide grating biosensor, an angular interrogation system for a resonant waveguide grating biosensor, a spatially scanned wavelength interrogation system, surface plasmon resonance system, surface plasmon resonance imaging, or a combination thereof.

13. The method of claim 1 wherein the dynamic mass redistribution signal comprises an optical signal that measures real-time kinetics of an effector cell contact-induced cellular response as a function of time.

14. The method of claim 1 wherein the dynamic mass redistribution signal comprises an optical signal that measures an endpoint or multiple points of an effector cell contact-induced cellular response over time and throughout an effector cell contact-induced event.

15. The method of claim 1 wherein the biosensor imaging system provides biosensor output comprising at least one of: the overall dynamics, the phase, the amplitude and kinetics of the phase, the transition time from one phase to another of the dynamic mass redistribution signal, or a combination thereof.

16. A method for characterizing a live cell-cell response to a stimulus, the method comprising:
providing a biosensor imaging system having a target cell immobilized on the biosensor;
contacting the immobilized target cell with a first stimulus;
contacting the first stimulus-contacted immobilized target cell with an effector cell;
detecting the dynamic mass redistribution of the target cell with the biosensor; and
determining the cell-signaling difference effect of contacting the stimulus- contacted target cell with the effector cell.

17. The method of claim 16 further comprising contacting with a second stimulus the first stimulus-contacted immobilized target cell and the effector cell.

18. The method of claim 16 wherein the second stimulus is the same as the first stimulus or the second stimulus is different from the first stimulus.

19. A method for characterizing a live cell-cell response to a stimulus, the method comprising:
providing a biosensor imaging system having a target cell and an effector cell immobilized on the biosensor's surface;
contacting the immobilized effector cell with a stimulus; and
detecting the dynamic mass redistribution signals of the target cell and the effector cell.

20. The method of claim 19 wherein the effector cell is a transmitter-releasing cell, the released transmitter activates the target cell and triggers signaling of the target cell.
